(19) United States
(12) Patent Application Publication
(10) Pub. No.: US 2011/0293713 A1
(43) Pub. Date: Dec. 1, 2011

(54) PHARMACEUTICAL FORMULATIONS COMPRISING NSAID AND PROTON PUMP INHIBITOR DRUGS

(76) Inventors: Snehalatha Movva, Hyderabad (IN); Hemanth Prakash Joshi, Gadag (IN); Rajan Kumar Verma, New Delhi (IN); Badri Narayanan Vishwanathan, Chennai (IN); Balakrishna Penugonda, Hyderabad (IN); Prasad Vure, Secunderabad (IN)

(21) Appl. No.: 13/149,354

(22) Filed: May 31, 2011

Related U.S. Application Data
(60) Provisional application No. 61/364,148, filed on Jul. 14, 2010.

(30) Foreign Application Priority Data
Jun. 1, 2010 (IN) 1507/CHE/2010

(51) Int. Cl. A61K 9/48; A61K 31/4439; A61P 1/00; A61K 9/20; A61P 29/00; A61P 9/00; A61K 9/14; A61K 31/225

(52) U.S. Cl. 424/451; 424/490; 514/338; 514/548; 424/464

(57) ABSTRACT

Aspects of the present application relate to pharmaceutical formulations comprising a nonsteroidal anti-inflammatory drug, together with a proton pump inhibitor drug, to reduce the incidence of gastrointestinal complications associated with chronic therapy with a nonsteroidal anti-inflammatory drug. Specific aspects of the application relate to fixed dose combinations comprising aspirin, or a derivative thereof, and omeprazole or esomeprazole, or pharmaceutically acceptable salts, solvates, or hydrates thereof.

Aspects of the present application relate to pharmaceutical formulations comprising a nonsteroidal anti-inflammatory drug, or a derivative thereof, together with a proton pump inhibitor drug, to reduce the incidence of gastrointestinal complications associated with chronic therapy with a nonsteroidal anti-inflammatory drug. Aspects of the present application further relate to fixed dose combinations comprising aspirin, or a derivative thereof, and a proton pump inhibitor drug, or pharmaceutically acceptable salts, solvates, or hydrates thereof.

Aspects of the present application provide methods for preventing vascular disorders such as myocardial infraction, stroke, unstable angina in patients with cardiovascular and cerebro-vascular risk, with low doses of aspirin, and simultaneously reducing the risk of gastrointestinal adverse effects normally associated with chronic aspirin therapy.

Aspirin, a nonsteroidal anti-inflammatory drug (NSAID), acts as an antiplatelet agent by inhibiting the cyclooxygenase-1 (COX-1) enzyme involved in platelet aggregation. It has a molecular formula C₉H₈O₄ and is represented by structural formula (1).

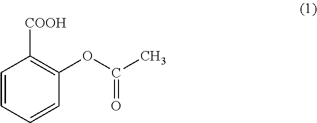

Unlike other NSAIDS, aspirin is a preferred NSAID for use as an antiplatelet agent, because of its long term inhibition of the COX-1 enzyme as compared with other NSAIDs. Long term prophylactic therapy with aspirin is frequently recommended in patients with cardiovascular and cerebro-vascular risks. However, even at low doses, aspirin is associated with a risk of gastrointestinal complications such as duodenal ulcer, peptic ulcer, bleeding, and perforation of gastrointestinal mucosa. Epidemiological evidence suggests that persons who take aspirin regularly (e.g., four or more days per week) are likely to suffer acute gastrointestinal bleeding or gastric ulceration.

One of the approaches for reducing the occurrence of gastrointestinal complications associated with chronic aspirin therapy is to concurrently administer ulcer protective or ulcer healing drugs. Suitable medications that address gastrointestinal complications with chronic aspirin therapy include H₂-receptor antagonists, proton pump inhibitors, and prostaglandin analogues.

Proton pump inhibitors (PPIs) are a class of acid-labile pharmaceutical compounds that block gastric acid secretion pathways. Exemplary proton pump inhibitors and their commercial products include, omeprazole (Prilosec®), lansoprazole (Prevacid®), esomeprazole (Nexium®), rabeprazole (Aciphex®), pantoprazole (Protonix®), pariprazole, tenatoprazole, and leminoprazole. The drugs of this class suppress gastrointestinal acid secretion by the specific inhibition of the H⁺/K⁺-ATPase enzyme system (proton pump) at the secretory surface of the gastrointestinal parietal cells. Most proton pump inhibitors are susceptible to acid degradation and, as such, are rapidly destroyed in an acidic pH environment of stomach. Therefore, proton pump inhibitors are often administered as enteric-coated dosage forms in order to permit release of the drug in the duodenum after having passed through the stomach. If the enteric-coating of these formulated products is disrupted or if a co-administered buffering agent fails to sufficiently neutralize the gastrointestinal pH, the uncoated drug is exposed to stomach acid and may be degraded.

Omeprazole, a substituted bicyclic aryl-imidazole having a chemical name 5-methoxy-2-[(4-methoxy-3,5-dimethyl-2-pyridinyl)methyl]sulfinyl]-1H-benzimidazole, is a proton pump inhibitor that inhibits gastrointestinal acid secretion. It is the active ingredient in products marketed as PRILOSEC® by AstraZeneca.

Esomeprazole was also developed and marketed by Astra Zeneca. It is the S-enantiomer of omeprazole and is the active ingredient in products marketed as NEXIUM®. It has improved efficacy over the racemic mixture of omeprazole. Esomeprazole is indicated in the treatment of dyspepsia, peptic ulcer disease, gastro-esophageal reflux disease, and Zollinger-Ellison syndrome. It has a molecular formula C₁₇H₁₈N₃O₃S and is represented by structural formula (2).

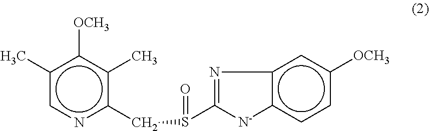

NEXIUM products are supplied as delayed-release capsules and in packets for a delayed-release oral suspension. Each delayed-release capsule contains 20 mg or 40 mg of esomeprazole (as esomeprazole magnesium trihydrate), in the form of enteric-coated granules. Also available is NEXIUM for delayed-release oral suspension containing 10 mg, 20 mg, or 40 mg of esomeprazole, in the form of the same enteric-coated granules used in NEXIUM delayed-release capsules, and also inactive granules. The esomeprazole granules and inactive granules are constituted with water to form a suspension and are given by oral, nasogastric, or gastric administration.

U.S. Patent Application Publication No. 2004/0121004 describes a non-enteric coated dosage form comprising a PPI, a buffer, and an NSAID. In the compositions disclosed, the NSAID and a PPI co-exist in a single dosage form without the need for an enteric coating covering the PPI. Further, the buffer provides immediate relief from gastric irritation and protects the stomach from local irritation sometimes caused simply by the presence of an NSAID in the stomach.

U.S. Patent Application Publication No. 2005/0249806 discloses pharmaceutical compositions comprising a PPI, one or more buffering agents, and a NSAID without any enteric coating. A buffering agent is used to prevent the acid degradation of PPI by sufficiently providing an alkaline microenvironment.

International Application Publication No. WO 97/25064 discloses an oral pharmaceutical composition comprising an acid sensitive PPI and one or more NSAIDs in a fixed formulation, wherein the proton pump inhibitor is protected by an enteric coating layer. The fixed formulation is in the form of an enteric coating layered tablet, a capsule, or a multiple unit tableted dosage form. The multiple unit dosage forms are most preferred.

U.S. Patent Application Publication No. 2002/0051814 relates to pharmaceutical compositions comprising omeprazole and aspirin, wherein the combination is useful for the treatment and prevention of cardiovascular events including heart attacks and platelet aggregation leading to a potential cardiac event. Both of the active ingredients are coated onto the same core.

There remains a need for pharmaceutically acceptable fixed dose combination formulations comprising aspirin and esomeprazole with minimized degradation of active in acidic environment, while providing maximum therapeutic benefits, as well as reducing the chances of frequent gastrointestinal adverse effects associated with chronic aspirin therapy.

### SUMMARY

Aspects of the present application relate to pharmaceutical formulations comprising aspirin or a derivative thereof, together with acid labile proton pump inhibitors to reduce the incidence of gastrointestinal complications associated with chronic aspirin therapy.

In embodiments, the application includes fixed dose combinations comprising aspirin and a proton pump inhibitor, wherein the total proton pump inhibitor drug-related impurity content is less than about 2%, or less than about 1%, of the label proton pump inhibitor drug content.

In embodiments, the application includes fixed dose combinations comprising aspirin and a proton pump inhibitor wherein the proton pump inhibitor component is enteric coated with suitable polymers, to prevent degradation in an acidic environment.

In embodiments, the application includes fixed dose combinations comprising aspirin and a proton pump inhibitor, wherein a proton pump inhibitor component is immediate release with suitable polymers.

In embodiments, the application includes pharmaceutical formulations comprising aspirin and a proton pump inhibitor, wherein degradation of the proton pump inhibitor is minimized by the incorporation of a stabilizing amount of an alkaline stabilizer.

In embodiments, the application includes pharmaceutical formulations containing about 5 mg to about 200 mg of a proton pump inhibitor and about 50 mg to about 700 mg of aspirin, per dosage unit.

In an aspect, the application includes methods of preparing pharmaceutical formulations of the present application.

In an aspect, the application includes methods of preventing vascular disorders such as myocardial infraction, stroke, and unstable angina in patients with cardiovascular and cerebrovascular risks, by administering low doses of aspirin and simultaneously reducing the risk of gastrointestinal adverse effects associated with chronic aspirin therapy.

### BRIEF DESCRIPTION OF THE DRAWINGS

FIG. 1 is a graphical representation of mean blood plasma concentrations of esomeprazole as a function of time, after administration of single doses of an esomeprazole 40 mg capsule to fasting subjects in Example 9.

FIG. 2 is a graphical representation of mean plasma concentrations of esomeprazole as a function of time, after administration of single doses of an esomeprazole 40 mg capsule to fed subjects in Example 9.

FIG. 3 is a graphical representation of mean plasma concentrations of aspirin as a function of time, after administration of single doses of an aspirin 325 mg tablet to fasting subjects in Example 9.

FIG. 4 is a graphical representation of mean plasma concentrations of aspirin as a function of time, after administration of single doses of an aspirin 325 mg tablet to fed subjects in Example 9.

FIG. 5 is a graphical representation of mean plasma concentrations of aspirin as a function of time, after administration of single doses of four aspirin 81 mg tablets to fasting subjects in Example 9.

FIG. 6 is a graphical representation of mean plasma concentrations of aspirin as a function of time, after administration of single doses of four aspirin 81 mg tablets to fed subjects in Example 9.

FIG. 7 is a graphical representation of mean plasma concentrations of salicylic acid as a function of time, after administration of single doses of an aspirin 325 mg tablet to fasting subjects in Example 9.

FIG. 8 is a graphical representation of mean plasma concentrations of salicylic acid as a function of time, after administration of single doses of an aspirin 325 mg tablet to fed subjects in Example 9.

FIG. 9 is a graphical representation of mean plasma concentrations of salicylic acid as a function of time, after administration of single doses of four aspirin 81 mg tablets to fasting subjects in Example 9.

FIG. 10 is a graphical representation of mean plasma concentrations of salicylic acid as a function of time, after administration of single doses of four aspirin 81 mg tablets to fed subjects in Example 9.

### DETAILED DESCRIPTION

Aspects of the present application relate to oral pharmaceutical formulations comprising aspirin or a derivative thereof, together with an acid labile proton pump inhibitor that reduces the incidence of gastrointestinal complications associated with chronic aspirin therapy. In embodiments, the present application relates to fixed dose pharmaceutical formulations comprising aspirin, or any pharmaceutically acceptable salts thereof, and a proton pump inhibitor drug, including any pharmaceutically acceptable salts thereof, for therapeutic purposes, and methods for preparing the formulations.

A dosage form of the present application improves patient compliance by combining the active ingredients in fixed dose formulations, while reducing the frequently observed gastrointestinal complications associated with chronic aspirin therapy.

A fixed dose combination formulation of the application provides methods of preventing vascular disorders such as myocardial infraction, stroke, and unstable angina in patients with cardiovascular and cerebrovascular risk, with aspirin and simultaneously reduces the risk of gastrointestinal adverse effects associated with chronic aspirin therapy.

In embodiments, aspirin may be present in an immediate release portion or an extended release portion, or in combinations of immediate and extended release portions.

In embodiments, the anti-ulcer drug is an acid-unstable proton pump inhibitor. The acid-unstable proton pump inhibitors used in the dosage forms of the invention may be present in their neutral form or in the form of their salts. Further, where applicable, the compounds may be used in a racemic form, in the form of a substantially pure enantiomer thereof, as mixtures of enantiomers in any proportions, or as salts thereof. Examples of proton pump inhibitors include omeprazole, esomeprazole, lansoprazole, pantoprazole, pariprazole, and laminoprazole. Esomeprazole and its salts are described herein to exemplify this class of drugs, and it is to be understood that other drugs from the class can be substituted therefor in the described formulations.

In embodiments, pellets, beads, or granules of aspirin may be prepared using techniques known in the art and are filled into capsules, together with a delayed release proton pump inhibitor portion.

In embodiments, a drug layer comprising aspirin may be coated onto a delayed release proton pump inhibitor portion, using coating techniques known in the art, and the coated material filled into capsules.

In embodiments, mini-tablets comprising aspirin may be prepared, using tableting techniques known in the art, and filled into capsules together with a controlled release proton pump inhibitor portion.

According to embodiments of the present application, dosage forms may be enteric coated multi-layered tableted systems, multi-unit particulate systems comprising a portion in the form of enterically coated delayed release particles, and another portion as immediate release, extended release, or combinations of immediate and extended release particles, filled into capsules or sachets.

In embodiments, a multi-particulate delayed release portion of the present application can be prepared by wet granulation, followed by extrusion and spheronization to obtain beads, pellets, or spheroids, which can be further coated with enteric polymers.

As used herein the term “aspirin” includes the compound aspirin, pharmaceutically acceptable salts of aspirin, isomers, solvates, complexes, and hydrates thereof, and any polymorphic crystalline or amorphous forms, including combinations thereof. Aspirin is used in the present application in the range of from 50 to 700 mg, or from 50 to 500 mg, or from 50 to 400 mg, or from 75 to 350 mg, per dosage unit.

As used herein the term “omeprazole” includes the compound omeprazole, pharmaceutically acceptable salts of omeprazole, solvates and hydrates thereof, and any polymorphic crystalline or amorphous form, including combinations thereof. Omeprazole is used in the present application in the range of from 5 to 200 mg, or from 10 to 60 mg, or from 20 to 40 mg, per dosage unit.

As used herein the term “esomeprazole” includes the compound esomeprazole, pharmaceutically acceptable salts of esomeprazole, solvates and hydrates thereof, and any polymorphic crystalline or amorphous form, including combinations thereof. Esomeprazole is used in the present application in the range of from 5 to 200 mg, or from 10 to 60 mg, or from 20 to 40 mg, per dosage unit.

As used herein, the terms “pharmaceutically acceptable salt,” or “salt,” include salts prepared using inorganic acids or bases, and organic acids or bases. Examples include metal salts such as aluminum, calcium, lithium, magnesium, potassium, sodium, and zinc salts. Examples of organic salt-forming bases include N,N′-dibenzylethylenediamine, chloroprocaine, choline, diethanolamine, ethylenediamine, meglumaine (N-methylglucamine), lysine, and procaine. Examples of salt-forming inorganic acids are hydrochloric, hydrobromic, hydroiodic, sulfuric, and phosphoric acids. Appropriate salt-forming organic acids include, for example, aliphatic, aromatic, carboxylic and sulfonic classes of organic acids, some examples of which are formic, acetic, propionic, succinic, glycolic, glucoronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic acids.

The foregoing lists are not intended to be exhaustive, and many other substances can be used.

As used herein the term “particulate” includes granules, spheroids, beads, pellets, and mini-tablets.

By “immediate-release”, it is meant a conventional or non-modified release form in which greater than or equal to about 75% of the active agent is released within about two hours after administration, or within about one hour after administration.

By “controlled-release” it is meant a dosage form in which the release of the active agent is controlled or modified over a period of time. Controlled can mean, for example, sustained, delayed, or pulsed-release at particular times following administration. Alternatively, “controlled” can mean that the release of the active agent is extended for longer times than would be observed for an immediate-release dosage form, i.e., at least over several hours after administration.

By “sustained-release” or “extended-release” is meant to include the release of the active agent at such a rate that steady state blood (e.g., plasma) levels are maintained within a therapeutic range, but below toxic levels, for at least about 8 hours, or at least about 12 hours, after administration. The term “steady-state” means that a plasma level for a given active agent has been achieved and which is maintained with subsequent doses of the drug at a level which is at or above the minimum effective therapeutic level, and is below the minimum toxic plasma level for a given active agent.

By “delayed-release”, it is meant that there is a time-delay after administration, before significant plasma levels of the active agent are achieved. A delayed-release formulation of the active agent can avoid an initial burst of the active agent, or can be formulated so that release of the active agent in the stomach is avoided and absorption is effected in the small intestine.

Certain formulations described herein may be “coated.” The coating can be a suitable coating, such as a functional or a non-functional coating, or multiple functional and/or non-functional coatings. By “functional coating” is meant to include a coating that modifies the release properties of the total formulation, for example, an enteric or sustained-release coating. By “non-functional coating” is meant to include a coating that is not a functional coating, for example, a cosmetic coating. A non-functional coating can have some impact on the release of the active agent by affecting the initial dissolution, hydration, perforation of the coating, etc., but would not be considered to be a significant deviation from the non-coated composition.

The term “enteric coating” may be defined as those which remain intact in stomach (and exhibit low permeability to gastric fluids), but break down readily once the dosage form reaches a higher pH environment such as the small intestine.

In further embodiments, the application includes oral fixed unit dose formulations such as enteric coated tablets, multilayered tablets, multiple-unit tableted dosage forms, capsules filled with enteric coated pellets, etc.

In embodiments, the solid dosage forms may be multilayered tableted systems comprising proton pump inhibitor-containing particles, coated with either enteric pH dependent release polymers or non-enteric time dependent release polymers for release in the small intestine.

In embodiments, a solid dosage form may be a multi-unit particulate system comprising one portion having delayed release particles and another portion having immediate release, extended release, or combinations of immediate and extended release particles, filled into capsules or sachets, or they may be tableted into finished dosage forms.

In embodiments, pharmaceutical combination products may comprise a proton pump inhibitor in a delayed release portion and aspirin in an immediate release, extended release, or combination of immediate and extended release particles, and further these discrete portions with different actives may be filled into capsules or sachets. In embodiments, pharmaceutical combination products may comprise a proton pump inhibitor in an immediate release portion and aspirin in a delayed release, extended release, or suitable combination of delayed and extended release particles, and further these discrete portions with different actives may be filled into capsules or sachets.

In embodiments, a delayed release portion of the present application may be prepared by coating powders, granules, pellets, tablets, or cores with one or more functional coatings, and they may be filled into capsules.

Pharmacologically inert pellets, beads, or cores that can be used include, but are not limited to: water-soluble particles such as sugar spheres, lactose, and the like; and water-insoluble particles such as celluloses, including microcrystalline cellulose, silicon dioxide, calcium carbonate, dicalcium phosphate anhydrous, dicalcium phosphate monohydrate, tribasic calcium phosphate, magnesium carbonate, magnesium oxide, and the like. An active substance may be layered onto inert particles or mixed with core-forming materials and made into a drug-containing core.

In embodiments, water soluble core materials such as sugar spheres may be coated with a seal coating layer. The purpose of sealing is to offer an initial protection and to prevent core ingredients from migrating into a coating, such as a drug-containing layer. Sealing may be accomplished by the application of polymer based coating materials onto the surface of the core particles. Examples of the polymers that can be used include, without limitation thereto, shellac, zein, hydroxypropyl methylcellulose (HPMC), polyvinyl acetate phthalate (PVAP), and cellulose acetate phthalate (CAP). This sealing agent may be dissolved in an appropriate aqueous or nonaqueous solvent.

In embodiments, a delayed release portion comprising esomeprazole magnesium may be prepared by coating a drug containing composition onto inert core materials or seal coated core materials. Drug layers may comprise acid labile esomeprazole together with at least one alkaline stabilizer, to minimize degradation of proton pump inhibitor in acidic environments.

Suitable alkaline stabilizers include, but are not limited to, sodium, potassium, calcium, magnesium and aluminum salts of phosphoric acid, carbonic acid, and citric acid, and aluminum/magnesium compounds such as Al₂O₃.6 MgO.CO₂.12H₂O and MgO.Al₂O₃.2SiO.nH₂O, where n is an integer of 2 or higher. In addition, the alkaline material may be an antacid material such as aluminum hydroxides, calcium hydroxides, magnesium hydroxides, and magnesium oxide. Alternatively, suitable alkaline stabilizers include basic amino acids and amino sugars such as 1-deoxy-1-(methylamino)-D-glucitol, commercially known as meglumine.

A drug layer may also comprise nonionic polyoxyethylene-polyoxypropylene copolymers as stabilizing or solubilizing agents. An example of a nonionic polyoxyethylene-polyoxypropylene copolymer according to one embodiment has an average molecular weight ranging from about 650 to about 9600.

In embodiments of the present application, drug loaded multi-particulates may be coated with enteric polymers. The term “enteric polymers” means polymeric substances that remain intact in the acidic environment of stomach, but decompose or solubilize readily once the dosage form reaches the neutral to alkaline environment of the small intestine. Various enteric coating material include, but are not limited to, cellulose derivatives such as cellulose acetate phthalates, cellulose acetate trimellitates, hydroxypropyl methylcellulose phthalates, and hydroxypropyl methylcellulose succinates, polyvinyl acetate phthalates, methacrylic acid-ethyl acrylate copolymers, such as poly(methacrylic acid, methyl methacrylate) 1:1, poly(methacrylic acid, ethyl acrylate) 1:1, poly(methacrylic acid, methyl methacrylate) 1:2, or combinations of enteric polymers. The enteric coating layer may be dispersed or dissolved in suitable aqueous or non-aqueous solvents. In embodiments, an alkaline neutralizer such as sodium hydroxide is added slowly to the enteric polymer dispersion, to avoid coagulation.

The enteric coating layer may further comprise suitable plasticizers to balance desired mechanical properties such as flexibility and hardness of the enteric coating layer. Example of such plasticizers include, but are not limited to, citric acid esters, phthalic acid esters, stearic acid esters, polyethylene glycols, polysorbates, dibutyl sebacate, cetyl alcohol, triacetin and combinations thereof. For example, embodiments use suitable combinations of triethyl citrate and glyceryl monostearate as plasticizers. The plasticizers may be dispersed in hot water and mixed with enteric coating polymer solutions or dispersions. The amount of plasticizer in an enteric coating layer may be optimized based on the particular enteric coating layer polymer chosen, and the amount of enteric polymer, so that the mechanical properties, i.e., flexibility and hardness of the enteric coating layer meet the desired requirements. The amount of plasticizer may vary from about 5 to about 15%, or about 8 to about 12%, or about 10%, by weight of the enteric coating layer.

In embodiments, an intermediate barrier coating layer is provided between the drug layer and enteric coating layer, to avoid instability problems associated with acid labile proton pump inhibitor being in contact with acidic enteric polymers. The materials which may be incorporated in the intermediate barrier layer are pharmaceutically acceptable materials such as sugar, polyethylene glycols, polyvinylpyrrolidones, polyvinyl alcohols, polyvinyl acetates, hydroxypropyl celluloses, methylcelluloses, ethylcelluloses, hydroxypropyl methylcelluloses, carboxymethyl cellulose sodium, water soluble salts of enteric coating polymers, and others, used alone or in mixtures. This intermediate barrier layer may comprise various other additives such as plasticizers, colorants, pigments, fillers, anti-tacking and antistatic agents (such as magnesium stearate, titanium dioxide, and talc), and other additives. Depending upon formulation needs, thicknesses of the intermediate barrier layer may be optimized. Intermediate barrier layers may also comprise alkaline stabilizers and serve to establish an alkaline pH microenvironment while in contact with physiological fluids.

An enteric coated delayed release portion may be coated with a composition comprising plasticizers, colorants, pigments, fillers, anti-tacking and anti sticking agents such as magnesium stearate, titanium dioxide, and other additives that may be included in order to impart a polishing effect. The thickness of this outer layer may be optimized depending upon the processing parameters useful for this application.

Suitable methods can be used to apply the coating layers. Processes such as simple or complex coacervation, interfacial polymerization, liquid drying, thermal and ionic gelation, spray drying, spray chilling, fluidized bed coating, pan coating, and electrostatic deposition may be used.

When a solvent is used to apply the coating, water and/or organic solvents that constitute a good solvent for the coating material, but are substantially non-solvents or poor solvents for the active agent, may be chosen. While the active agent may partially dissolve in the solvent, typically the active ingredient will precipitate out of the solvent during a drying step more rapidly than the coating material. Representative solvents include water, an alcohol such as methanol, ethanol, and isopropyl alcohol, methylene chloride, a halogenated hydrocarbon such as dichloromethane (methylene chloride), a hydrocarbon such as cyclohexane, and combinations comprising any one or more of the foregoing solvents.

The concentrations of polymer in the solvent will normally be less than about 75% by weight, and typically about 10 to about 30% by weight. After coating, the coated dosage forms may be allowed to cure for at least about 1 to about 2 hours at temperatures about 35° C. to about 60° C., or about 40° C.

Pharmacokinetic properties of drug products can be studied by administering the products to healthy volunteer subjects. Serum plasma samples are obtained at regular intervals following dosing and assayed for drug (or sometimes metabolite) concentrations. For a pharmacokinetic comparison, the following parameters can be calculated:

AUCₒ₋ₜ=the area under plasma concentration versus time curve, from the time of administration to the last measurable concentration.

AUC_{0-∞}=area under the plasma concentration versus time curve, from the time of administration to infinity.

Cₘₐₓ=maximum plasma concentration.

Tₘₐₓ=Time after dosing until the maximum measured plasma concentration.

In embodiments, mini-tablets comprising aspirin may be prepared using typical tableting techniques and filled into hard gelatin capsules, such as size 0 or size 1, together with a controlled release esomeprazole portion.

In embodiments, the application includes fixed dose combinations comprising aspirin and a proton pump inhibitor, wherein the total proton pump inhibitor drug related impurity content is less than about 2%, or less than about 1%, of the label proton pump inhibitor drug content.

Esomeprazole and its drug-related impurities can be analyzed using a HPLC method with a 4.6×150 mm column containing a 3.5 μm packing of octylsilane, chemically bonded to porous silica or ceramic micro-particles (e.g., a 150×4.6 mm Xterra, RP 8, 3.5 μm or equivalent column), where the liquid chromatograph is equipped with a 305 nm UV detector, having a column temperature of 25° C., at a 1 mL per minute flow rate and with a run time of 60 minutes.

Structures for certain of the potential impurity compounds related to esomeprazole are shown below.

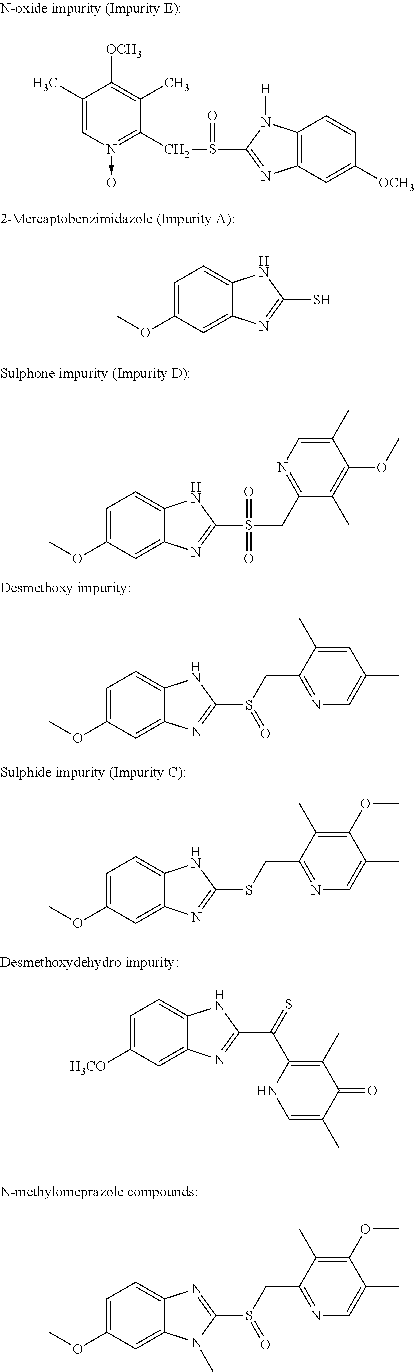

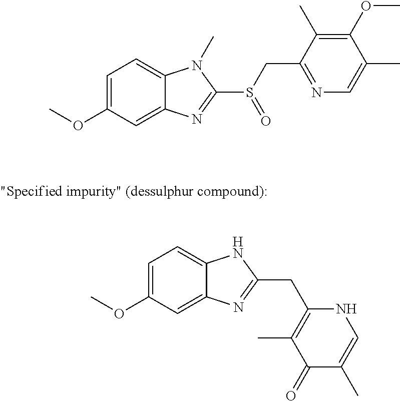

The total of proton pump inhibitor drug-related impurities contained in pharmaceutical formulations of the application generally will be less than about 3% of the label proton pump inhibitor drug content. In embodiments, the total proton pump inhibitor drug-related impurity content will be less than about 2%, or less than about 1%, of the label proton pump inhibitor drug content.

In embodiments, pharmaceutical formulations of the present application optionally contain additives additional to the active agents, which include without limitation any one or more of diluents, binders, disintegrants, surfactants, and other additives that are commonly used in solid dosage form preparations.

### Diluents

Various useful fillers or diluents include, but are not limited to, starches, lactose, cellulose derivatives, confectioner's sugar, and the like. Different grades of lactose include, but are not limited to, lactose monohydrate, lactose DT (direct tableting), lactose anhydrous, FLOWLAC™ (available from Meggle Products), PHARMATOSE™ (available from DMV), and others. Different starches include, but are not limited to, maize starch, corn starch (commercially available as Corn Starch B700), potato starch, rice starch, wheat starch, pregelatinized starch (commercially available as PCS PC10 from Signet Chemical Corporation), starch 1500 and starch 1500 LM (low moisture content grade) from Colorcon, fully pregelatinized starch (commercially available as National 78-1551 from Essex Grain Products), and others. Different cellulose materials that can be used include crystalline celluloses and powdered celluloses. Examples of crystalline cellulose products include but are not limited to CEOLUS™ KG801, AVICEL™ PH101, PH102, PH301, PH302, PH-F20, PH-112, microcrystalline cellulose 114, and microcrystalline cellulose 112. Other useful diluents include but are not limited to carmellose, sugar alcohols such as mannitol (e.g., PEARLITOL™ SD200), sorbitol and xylitol, calcium carbonate, magnesium carbonate, dibasic calcium phosphate, and tribasic calcium phosphate.

### Binders

Various useful binders include, but are not limited to, hydroxypropylcelluloses, also called HPC (e.g., KLUCEL™ LF or EXF) and useful in various grades, hydroxypropyl methylcelluloses, also called hypromelloses or HPMC (e.g., METHOCEL™) and useful in various grades, polyvinylpyrrolidones or povidones (such as grades PVP-K25, PVP-K29, PVP-K30, and PVP-K90), PLASDONE™ S 630 (copovidone), powdered acacia, gelatin, guar gum, carbomers (e.g., CARBOPOL™) methylcelluloses, polymethacrylates, and starches.

### Disintegrants

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, KOLLIDON™ CL (from BASF in Germany), POLYPLASDONE™ XL, XI-10, and INF-10 (from ISP Inc., USA), and low-substituted hydroxypropylcelluloses. Examples of low-substituted hydroxypropylcelluloses include, but are not limited to, low-substituted hydroxypropylcelluloses LH11, LH21, LH31, LH22, LH32, LH20, LH30, LH32 and LH33 (from Shin-Etsu Chemical Co., Ltd.). Other useful disintegrants include sodium starch glycolate, colloidal silicon dioxide, and starches.

### Lubricants

An effective amount of any pharmaceutically acceptable tableting lubricant can be added to assist with compressing tablets. Useful tablet lubricants include magnesium stearate, glyceryl monostearate, palmitic acid, talc, carnauba wax, calcium stearate sodium, sodium or magnesium lauryl sulfate, calcium soaps, zinc stearate, polyoxyethylene monostearate, calcium silicate, silicon dioxide, hydrogenated vegetable oils and fats, stearic acid, and combinations thereof.

Certain specific aspects and embodiments of the application will be explained in more detail with reference to the following examples, being provided only for purposes of illustration, and it is to be understood that the present disclosure is not to be limited thereto.

### Examples 1-2

### Esomeprazole Delayed Release Pellets

| | mg/Capsule | |
|---|---|---|
| Ingredient | 1 | 2 |

I. Seal Coated Cores

| | | |
|---|---|---|
| Sugar spheres (45-60 mesh) | 19 | 19 |
| HPMC 5 cps | 1 | 1 |
| Water* | q.s. | q.s. |

II. Drug Layer

| | | |
|---|---|---|
| Esomeprazole magnesium | 41.4 | 41.4 |
| HPMC 5 cps | 10 | 10 |
| Meglumine | 2.5 | 2.5 |
| Poloxamer 188 | 4 | 4 |
| Methanol* | q.s. | q.s. |
| Methylene chloride* | q.s. | q.s. |

III. Intermediate Barrier Layer

| | | |
|---|---|---|
| HPMC 5 cps | 5.7 | 5.7 |
| Talc | 9.8 | 9.8 |
| Magnesium stearate | 0.8 | 0.8 |
| Isopropyl alcohol* | q.s. | q.s. |
| Methylene chloride* | q.s. | q.s. |

IV. Enteric Coating Layer

| | | |
|---|---|---|
| Methacrylic acid copolymer (Eudragit L 30 D 55) | 62.6 | 78.2 |
| Triethyl citrate | 6.2 | 7.8 |
| Glyceryl monostearate | 1.3 | 1.6 |
| Talc | 7.7 | 9.7 |
| Sodium hydroxide | 0.3 | 0.4 |
| Water* | q.s. | q.s. |

V. Polishing

| | | |
|---|---|---|
| Glyceryl monostearate | 0.2 | 0.3 |
| Talc | 0.5 | 0.5 |
| Isopropyl alcohol* | q.s. | q.s. |

*Evaporates during processing.
**Eudragit L 30 D 55 is poly(methacrylic acid, ethyl acrylate 1:1), supplied by Evonik Industries, Germany.

Manufacturing Procedure:

1. Sugar spheres passing through a 45 mesh sieve and retained on a 60 mesh sieve are coated with HPMC solution in water, then dried.

2. A drug layer dispersion is prepared by dissolving esomeprazole magnesium in a previously prepared solution of meglumine and poloxamer in the solvent system containing methanol and methylene chloride, the system being maintained at 2-10° C.

3. The seal coated sugar spheres are coated with the drug layer dispersion, using a fluid bed processor (FBP), and dried.

4. An intermediate coating dispersion is prepared by dissolving HPMC in part of the isopropyl alcohol and methylene chloride solvent system. Talc and magnesium stearate are combined with the remaining solvent system and passed through a colloid mill. The solution and dispersion are mixed.

5. Drug loaded pellets of step 3 are coated with intermediate coating dispersion in a fluid bed processor, and dried.

6. Enteric coating is prepared by slowly adding a sodium hydroxide solution to a Eudragit L 30 D 55 dispersion, avoiding polymer coagulation. Glyceryl monostearate and talc are mixed with hot water and then added to the Eudragit dispersion.

7. Pellets from step 5 are coated with the enteric coating dispersion, and dried.

8. Polishing of the pellets is done in a FBP, using a glyceryl monostearate and talc dispersion.

9. Pellets corresponding to 40 mg of esomeprazole are filed into capsules.

### Example 3

### Esomeprazole Immediate Release Pellets

| | |
|---|---|
| Ingredient | mg/Capsule |

Seal Coating

| | |
|---|---|
| Sugar spheres (45-60 mesh) | 19 |
| HPMC 5 cps | 1 |
| Water* | q.s. |

Drug Layer

| | |
|---|---|
| Esomeprazole magnesium | 22.9 |
| HPMC 5 cps | 10 |
| Povidone (K30) | 5 |
| Macrogol/PEG 400 | 4 |
| Poloxamer 188 | 3 |
| Croscarmellose sodium | 4 |
| Lactose monohydrate | 23 |
| Magnesium oxide light | 5 |
| Methanol* | q.s. |
| Water* | q.s. |

Protective Coating

| | |
|---|---|
| Hypromellose 3 cPs | 4 |
| Lactose monohydrate | 12 |
| Croscarmellose sodium | 5.5 |
| Macrogol/PEG 400 | 0.5 |
| Water* | q.s. |

*Evaporates during processing.

Manufacturing Procedure:

1. Sugar spheres are coated with HPMC solution in water, then dried.

2. Drug coating dispersion is prepared by dissolving esomeprazole in a previously prepared dispersion of magnesium oxide light, poloxamer, HPMC, povidone, PEG 400, croscarmellose sodium, and lactose monohydrate. The solvent system is methanol and water in a 90:10 by volume ratio and the temperature of the dispersion is maintained at 2-10° C.

3. The seal coated sugar spheres are coated with the drug dispersion, using a fluid bed processor, and then are dried.

4. A protective coating dispersion is prepared by dissolving HPMC and PEG 400 in water. To this, lactose monohydrate and croscarmellose sodium are added with stirring.

5. Protective coating ingredients are mixed and applied to the drug-coated pellets, using a FBP, and the pellets are dried.

### Examples 4-5

### Aspirin Immediate Release Tablets

| | mg/Tablet | |
|---|---|---|
| Ingredient | 4 and 4A | 5 and 5A |
| Aspirin* | 325 | 81 |
| Powdered cellulose | 23 | 5.7 |
| Corn starch B700 | 30 | 7.4 |
| Colloidal silicon dioxide | 2 | 1 |
| Talc | 3 | 0.7 |
| Opadry AMB OY 29000** | 11.5 | 3.3 |

*Examples 4 and 5 use Rhodine ® aspirin 3040 grade. Examples 4A and 5A use Rhodine ® aspirin 3020 grade.
**Opadry AMB OY 29000 contains partially hydrolyzed polyvinyl alcohol, talc, soya lecithin, and xanthan gum, and is supplied by Colorcon.

Manufacturing Procedure:

1. Aspirin is mixed with powdered cellulose, corn starch, and colloidal silicon dioxide. Talc is added and the lubricated blend is compressed into tablets containing 325 mg of aspirin, using 12×5 mm capsule-shaped punches. A portion of the lubricated blend also is compressed into tablets containing 81 mg of aspirin, using 5.5 mm round-shaped punches.

2. Tablets are coated with a dispersion of Opadry AMB OY 29000, then dried.

### Examples 6-7

### Aspirin Delayed Release Tablets

| | mg/Tablet | |
|---|---|---|
| Ingredient | 6 | 7 |
| Aspirin | 325 | 81 |
| Powdered cellulose | 23 | 5.7 |
| Corn starch B700 | 30 | 7.4 |
| Colloidal silicon dioxide | 2 | 1 |
| Talc | 3 | 0.7 |

Enteric Coating

| | | |
|---|---|---|
| Eudragit L30 D55 | 49.63 | 12.36 |
| Talc | 9.93 | 2.47 |
| Triethyl citrate | 4.96 | 1.23 |
| Sodium hydroxide | 0.24 | 0.06 |
| Water* | q.s. | q.s. |

*Evaporates during processing.

Manufacturing Procedure:

1. Aspirin is mixed with powdered cellulose, corn starch, and colloidal silicon dioxide. Talc is added and the lubricated blend is compressed into tablets containing 325 mg of aspirin, using 12×5 mm capsule-shaped punches. A portion of the lubricated blend also is compressed into tablets containing 81 mg of aspirin, using 5.5 mm round-shaped punches.

2. Eudragit L30 D55 dispersion is mixed with triethyl citrate.

3. Talc is passed through a #60 mesh sieve and dispersed in a small quantity of water using a colloid mill, and the dispersion is added to the Eudragit dispersion.

4. Sodium hydroxide is dissolved in water and the solution is slowly added to the Eudragit dispersion, without forming agglomerates.

5. The dispersion is coated onto the tablets, using a coating pan and maintaining product temperature at 28-32° C.

6. Coated tablets are cured for 1 hour at low rotational speed, maintaining the temperature at 32-35° C.

### Example 8

### Encapsulated Products

A. Pellets corresponding to 40 mg esomeprazole, prepared in Examples 1 or 2, and a 325 mg aspirin tablet, prepared in Examples 4 or 4A, are filled into size 0 capsules.

B. Pellets corresponding to 40 mg of esomeprazole, prepared in Examples 1 or 2, and four 81 mg aspirin tablets, prepared in Examples 5 or 5A, are filled into a size 0 capsule.

### Example 9

### Pharmacokinetic Study

Products of Examples 1 and 2, the commercial product NEXIUM 40 mg capsules (“Reference” for esomeprazole results), products of Examples 4, 4A, 5, and 5A, and the commercial product Genuine Bayer® Aspirin 325 mg tablets (“Reference” for aspirin results) are administered in single doses in a 3-way crossover human pharmacokinetic study to 21 subjects, under both fasting and fed conditions. The mean pharmacokinetic data from plasma analyses are tabulated below.

| | | | | |
|---|---|---|---|---|
| | AUC₍₀₋ₜ₎ | AUC_{(0-∞)} | | |
| Product | (ng · hr/mL) | (ng · hr/mL) | Cₘₐₓ (ng/mL) | Tₘₐₓ (hours) |

40 mg Esomeprazole DR Capsule (Fasting)-Esomeprazole Results

| | | | | |
|---|---|---|---|---|
| Reference | 6865 | 6782 | 1910 | 2.33 |
| Example 1 | 6200 | 6228 | 1780 | 2.33 |
| Example 2 | 6435 | 6468 | 1874 | 2.33 |

40 mg Esomeprazole DR Capsule (Fed)-Esomeprazole Results

| | | | | |
|---|---|---|---|---|
| Reference | 5158 | 5220 | 1072 | 4.50 |
| Example 1 | 4355 | 4394 | 852 | 5.00 |
| Example 2 | 4968 | 5009 | 1065 | 5.00 |

325 mg Aspirin Tablets (Fasting) Aspirin Results

| | | | | |
|---|---|---|---|---|
| Reference | 4610 | 4711 | 4241 | 0.92 |
| Example 4 | 4478 | 4567 | 4520 | 0.83 |
| Example 4A | 4447 | 4529 | 3976 | 1.04 |

325 mg Aspirin Tablets (Fed)-Aspirin Results

| | | | | |
|---|---|---|---|---|
| Reference | 5383 | 5497 | 3506 | 1.38 |
| Example 4 | 5338 | 5456 | 3933 | 1.50 |
| Example 4A | 5178 | 5291 | 3852 | 1.25 |

Four 81 mg Aspirin Tablets (Fasting)-Aspirin Results

| | | | | |
|---|---|---|---|---|
| Reference | 4016 | 4125 | 2697 | 0.75 |
| Example 5 | 4214 | 4279 | 3149 | 0.75 |
| Example 5A | 4133 | 4214 | 3047 | 0.75 |

Four 81 mg Aspirin Tablets (Fed)-Aspirin Results

| | | | | |
|---|---|---|---|---|
| Reference | 3845 | 3870 | 2050 | 2 |
| Example 5 | 3900 | 3924 | 2108 | 1.5 |
| Example 5A | 3991 | 4012 | 2015 | 1.88 |

325 mg Aspirin Tablets (Fasting)-Salicylic Acid Results

| | | | | |
|---|---|---|---|---|
| Reference | 129820.04 | 132813.41 | 25663.29 | 2.00 |
| Example 4 | 132107.40 | 135327.24 | 27075.00 | 2.00 |
| Example 4A | 125444.95 | 129329.38 | 24927.91 | 2.67 |

325 mg Aspirin Tablets (Fed)-Salicylic Acid Results

| | | | | |
|---|---|---|---|---|
| Reference | 110103 | 115640 | 23094 | 2.50 |
| Example 4 | 113049 | 118174 | 24734 | 2.25 |
| Example 4A | 110431 | 116162 | 23463 | 2.25 |

Four 81 mg Aspirin Tablets (Fasting)-Salicylic Acid Results

| | | | | |
|---|---|---|---|---|
| Reference | 127301 | 131170 | 23381 | 2.33 |
| Example 5 | 127697 | 132370 | 23896 | 2.33 |
| Example 5A | 129045 | 133823 | 23667 | 2.50 |

Four 81 mg Aspirin Tablets (Fed)-Salicylic Acid Results

| | | | | |
|---|---|---|---|---|
| Reference | 116598 | 126944 | 21992 | 3 |
| Example 5 | 122217 | 130957 | 23371 | 2.75 |
| Example 5A | 119841 | 126956 | 23424 | 2.88 |

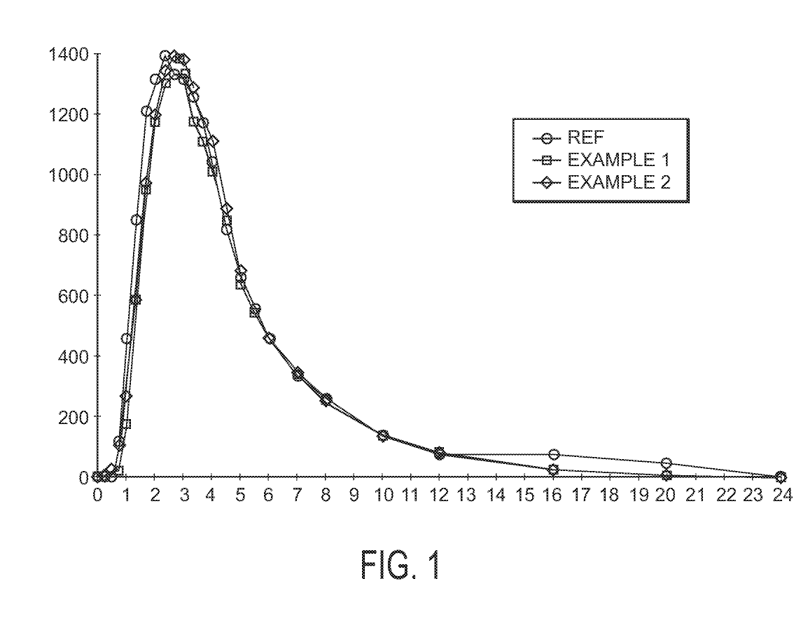

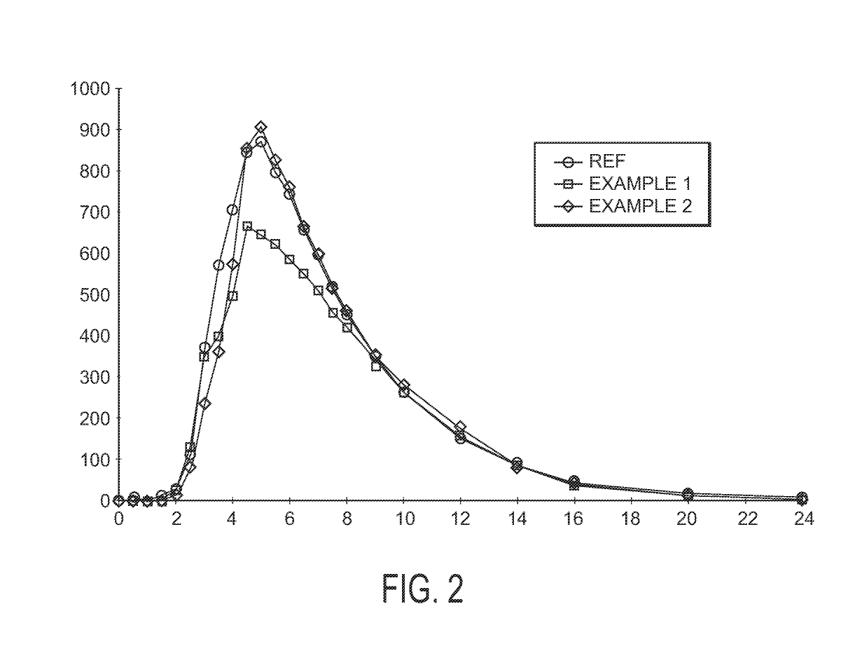

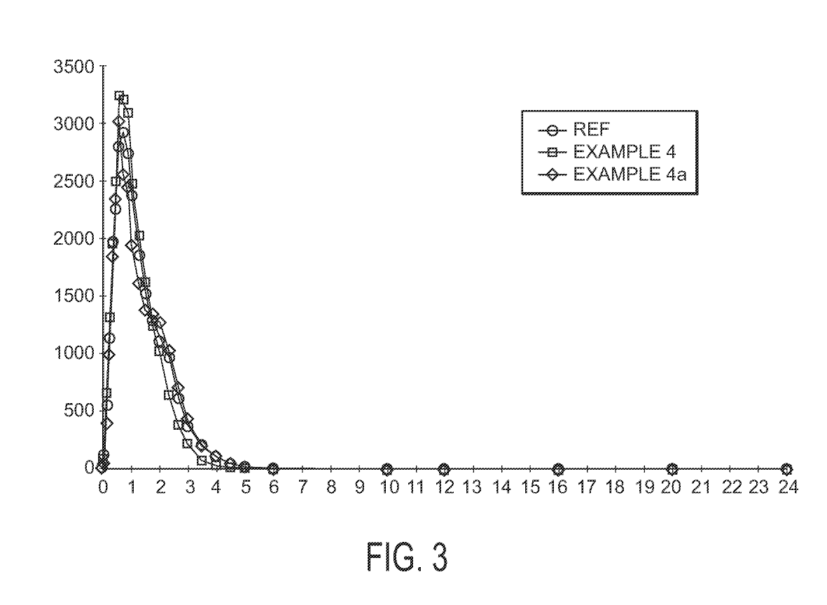

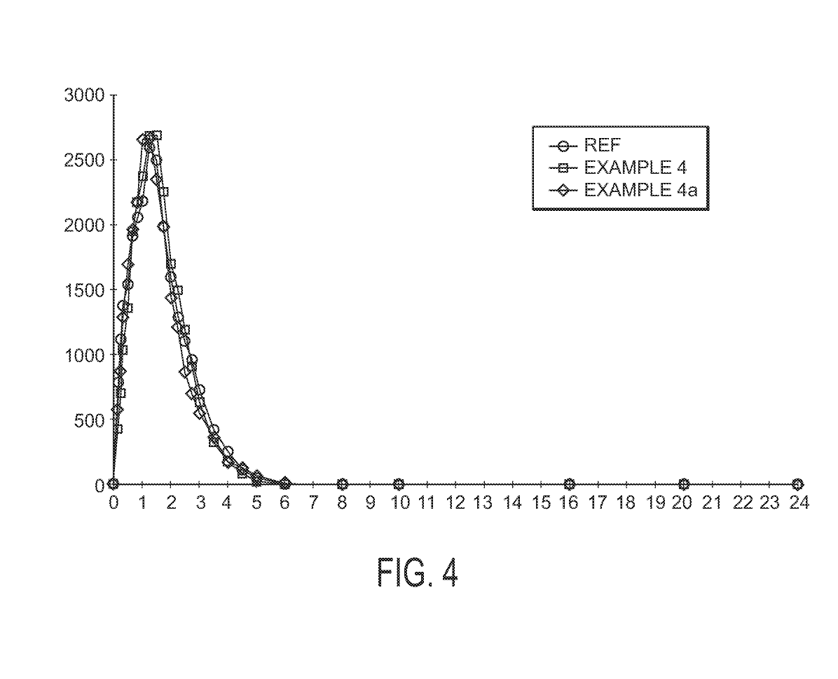

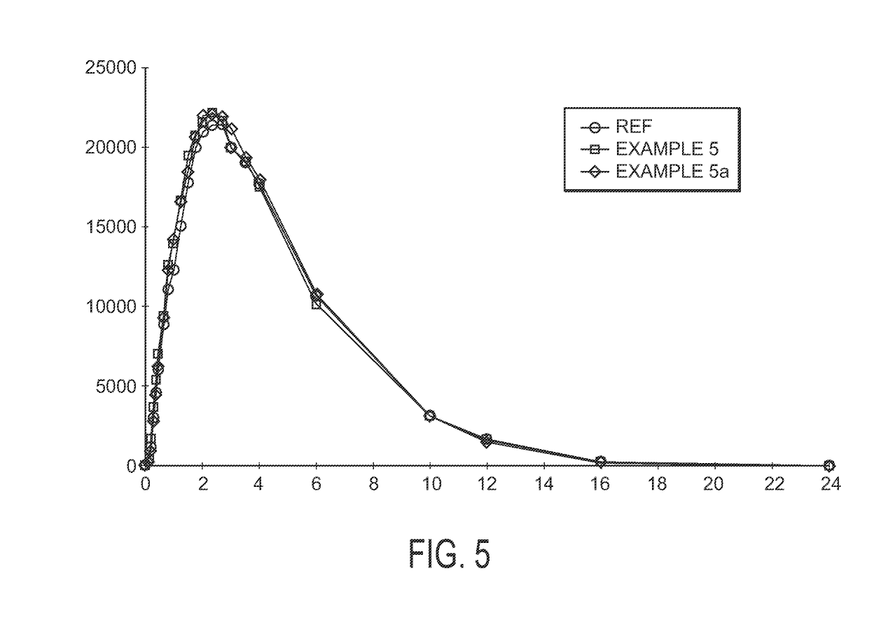

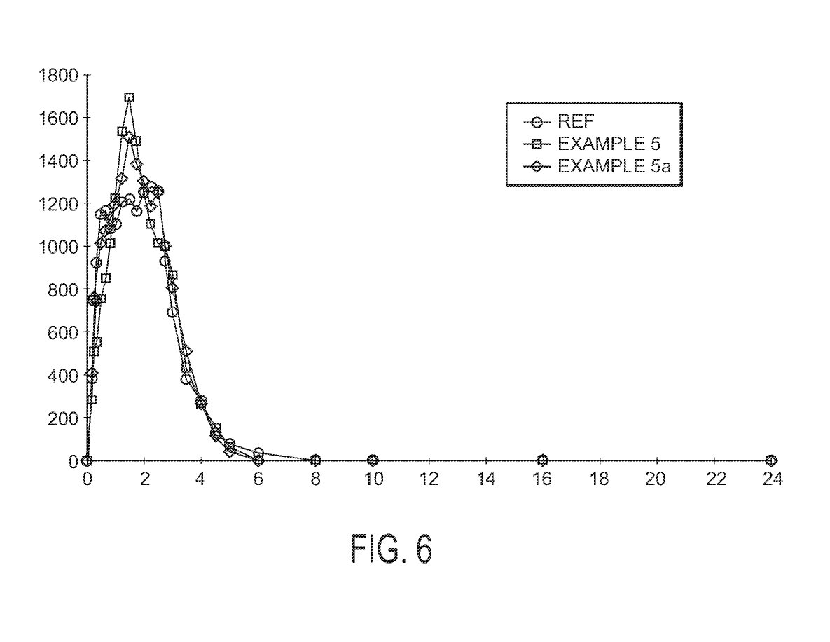

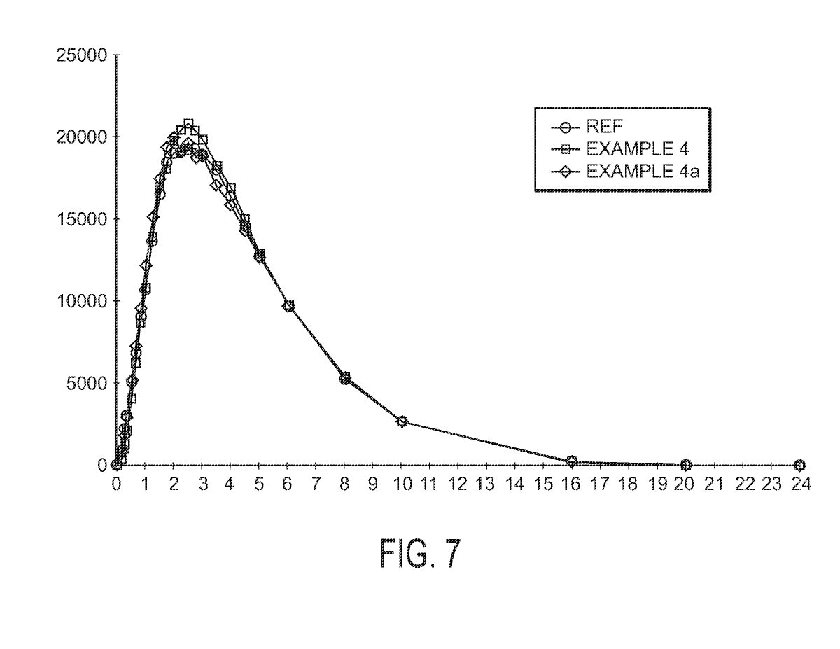

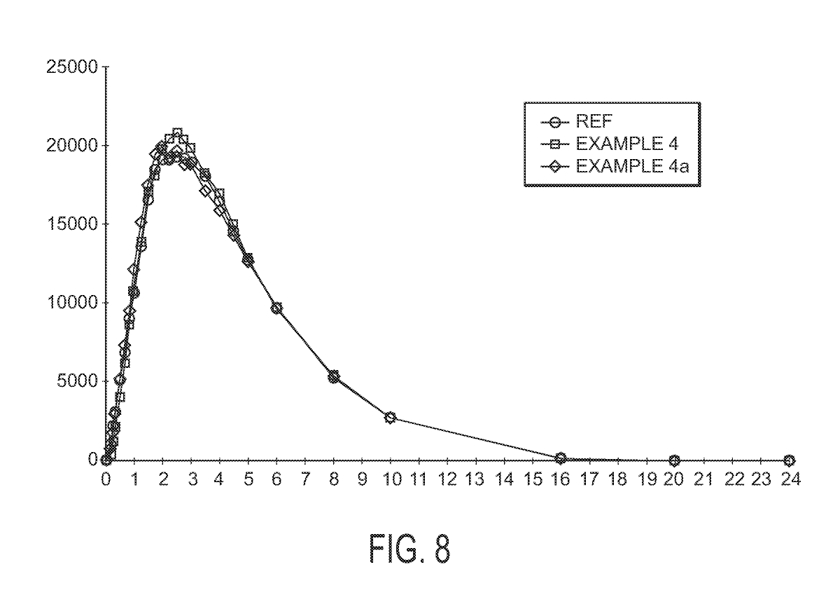

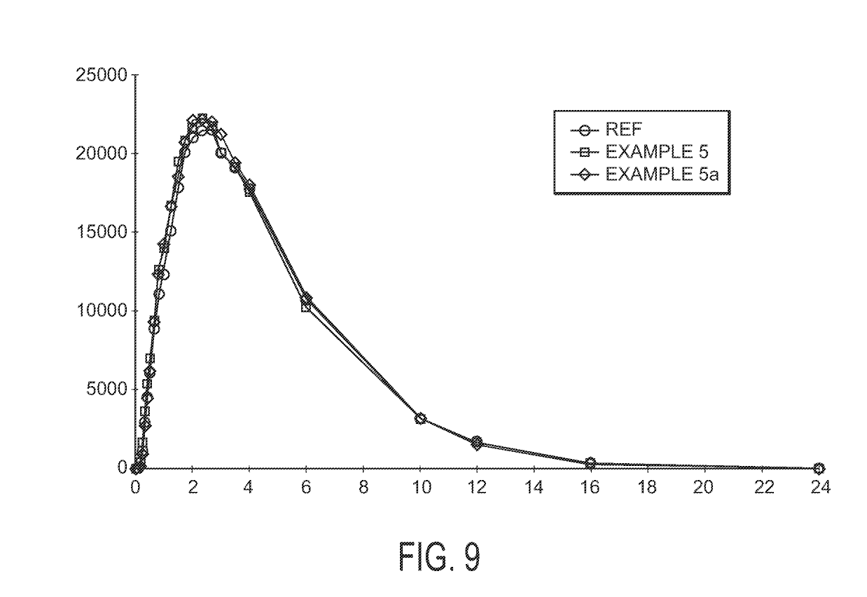

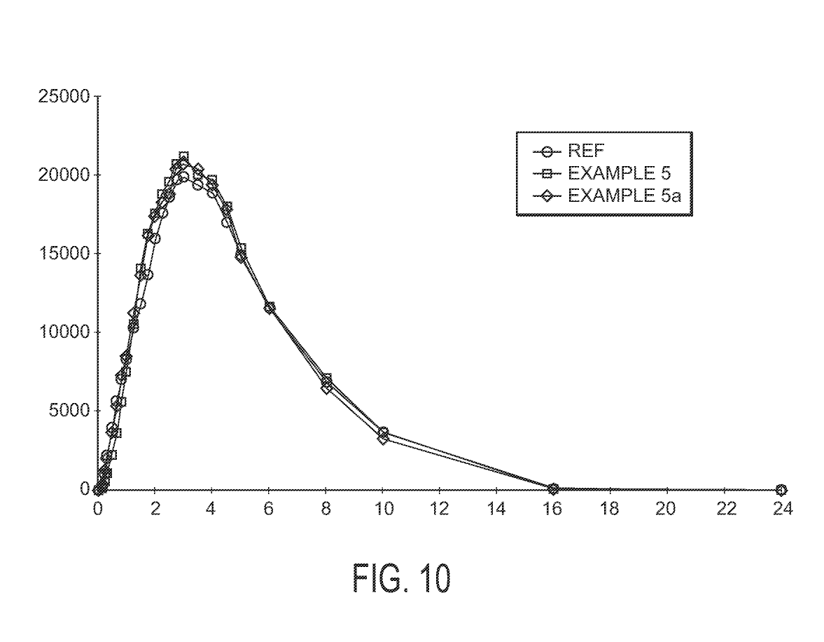

FIGS. 1-10 provide graphical depictions of these pharmacokinetic study results, where drug plasma concentrations are plotted as a function of time, after administration. In the figures, the x-axis values are hours and the y-axis values are mean concentrations, in ng/mL, of the compound being analyzed.

### Example 7

### Stability Studies

Capsules, filled with esomeprazole-containing pellets of Example 1 and aspirin delayed release tablets of Example 4, are packaged in closed HDPE containers, each provided with a 2 g molecular sieve desiccant pouch, and stored at 40° C. and 75% relative humidity for two months. Samples are analyzed for their impurity profiles and dissolution characteristics, initially and after storage, and results are shown in the tables below, where drug assay and impurity amounts are expressed as percentages of the label drug content.

### Esomeprazole Capsules

| Parameter | | Initial | 1 Month | 2 Months |
|---|---|---|---|---|
| Drug Assay | | 98.6 | — | 99.4 |
| Drug-Related | Desmethyl dehydro | 0.01 | 0.04 | 0.07 |
| Impurity | Specified impurity | 0.01 | 0.02 | — |
| | (Dessulphur) | | | |
| | Benzimidazole (Imp. A) | 0.01 | 0.02 | 0.07 |
| | Sulphone (Imp. D) | 0.01 | 0.03 | 0.02 |
| | N-methylomeprazole | 0.01 | 0.05 | 0.08 |
| | Sulphide (Imp. C) | 0.01 | 0.01 | 0.02 |
| | N-oxide (Imp. E) | — | — | 0.00 |
| | Desmethoxy | — | 0.01 | 0.02 |
| | Highest Unidentified | 0.01 | 0.03 | 0.08 |
| | Total | 0.1 | 0.29 | 0.52 |
| Cumulative % | 120 minutes | 0 | 0 | — |
| of | 125 minutes | 22 | 20 | — |
| Esomeprazole | 130 minutes | 54 | 50 | — |
| Released* | 135 minutes | 80 | 75 | — |
| | 150 minutes | 96 | 95 | — |
| | 180 minutes | 93 | 92 | — |

*Dissolution study is conducted using a USP type 2 apparatus, rotated at 100 rpm, in 300 mL of 1.2N HCl medium for the first 120 minutes, then in 1000 mL of pH 6.8 sodium phosphate buffer medium.

### Aspirin Tablets

| Parameter | | Initial | 1 Month | 2 Months |
|---|---|---|---|---|
| Drug Assay | | 99.7 | — | 100 |
| Drug-Related | Salicylic Acid | 0.20 | 0.25 | 0.32 |
| Impurity | | | | |
| Cumulative % | 5 minutes | 38 | — | 36 |
| of Aspirin | 10 minutes | 93 | — | 86 |
| Released* | 15 minutes | 99 | — | 98 |
| | 30 minutes | 99 | — | 99 |
| | 60 minutes | 98 | — | 98 |

*Dissolution study is conducted in 900 mL of pH 4.5 acetate buffer medium, using a USP type 2 apparatus rotated at 100 rpm.

We claim:

1. A pharmaceutical dosage form for oral administration, comprising:
a) one or more enteric coated particles containing a proton pump inhibitor drug or a salt thereof; and
b) one or more particles containing acetylsalicylic acid that are not enteric coated;
wherein the total amount of proton pump inhibitor drug-related impurities is less than about 2 percent, or less than about 1 percent, of the label content of the proton pump inhibitor drug.

2. The dosage form of claim 1, wherein the total amount of proton pump inhibitor drug-related impurities is less than about 1 percent of the label content of the proton pump inhibitor drug.

3. The dosage form of claim 1, wherein a proton pump inhibitor drug is omeprazole or a salt thereof.

4. The dosage form of claim 1, wherein a proton pump inhibitor drug is esomeprazole or a salt thereof.

5. The dosage form of claim 1, wherein a particle of b) is a tablet.

6. The dosage form of claim 1, wherein particles of a) and particles of b) are contained in a capsule.

7. The dosage form of claim 1, wherein particles of a) are pellets.

8. The dosage form of claim 1, wherein particles of a) and particles of b) are formed into a tablet.

9. The dosage form of claim 1, comprising esomeprazole in amounts from 5 mg to 200 mg and acetylsalicylic acid in amounts from 50 mg to 700 mg.

10. A method for treating inflammation, comprising administering the dosage form of claim 1.

11. A method of preventing thromboembolic vascular events, and/or reducing gastrointestinal complications associated with acetylsalicylic acid treatment, comprising administering the dosage form of claim 1.

12. A pharmaceutical dosage form for oral administration, comprising:
a) one or more enteric coated particles containing omeprazole or esomeprazole, optionally present as a salt; and
b) one or more particles containing acetylsalicylic acid that are not enteric coated;
wherein the total amount of omeprazole- or esomeprazole-related impurities is less than about 2 percent, or less than about 1 percent, of the label content of the proton pump inhibitor drug.

13. The dosage form of claim 12, wherein particles of a) are pellets.

14. The dosage form of claim 12, wherein a particle of b) is a tablet.

15. The dosage form of claim 12, wherein particles of a) are pellets and a particle of b) is a tablet, both of a) and b) being contained in a capsule.

16. A process for manufacturing a pharmaceutical dosage form for oral administration, comprising:
a) preparing enteric coated pellets containing a proton pump inhibitor drug;
b) preparing tablets containing acetylsalicylic acid; and
c) filling pellets of a) and one or more tablets of b) into a capsule.

17. The process according to claim 16, wherein a proton pump inhibitor drug is omeprazole or a salt thereof.

18. The process according to claim 16, wherein a proton pump inhibitor drug is esomeprazole or a salt thereof.
